(19) United States
(12) Patent Application Publication
(10) Pub. No.: US 2024/0249813 A1
(43) Pub. Date: Jul. 25, 2024

(54) Activity Recommendation Based on Biometric Data from Wearable Device

(71) Applicant: Analogy Partners, LLC, Skillman, NJ (US)

(72) Inventors: John Anthony Singarayar, Skillman, NJ (US); Shanil Singarayar, Skillman, NJ (US); Ganepola Arachchige Dilshan Maduranga Ganepola, Kiribathgoda (LK); Appuhamilage Dilshan Nipuna Wickramarachchi, Ganemulla (LK); Gunawardana Mudiyanselage Sanduni Nimeshika Gunawardana, Pahala Kurukuttala (LK)

(73) Assignee: Analogy Partners, LLC, Skillman, NJ (US)

(21) Appl. No.: 18/404,566

(22) Filed: Jan. 4, 2024

Related U.S. Application Data
(60) Provisional application No. 63/440,001, filed on Jan. 19, 2023.

(51) Int. Cl. G16H 20/30; A61B 5/16

(52) U.S. Cl. CPC G16H 20/30; A61B 5/165

(57) ABSTRACT

A method for recommending activities to decrease stress. The method may include monitoring biometric data regarding a user, the biometric data may be captured by a wearable sensor worn by the user; identifying a stress response level based on a comparison of the monitored biometric data to data from a cohort having one or more similarities to the user; generating an activity recommendation based on a speed, degree, and/or duration of one or more changes to the stress response level; and sending a notification to a user device of the user regarding the identified stress response level in real-time, wherein the notification may include the activity recommendation.

### RELATED APPLICATIONS

This application is related and claims priority to U.S. Provisional Patent Application No. 63/440,001, filed on Jan. 19, 2023 the application of which is incorporate herein by reference in its entirety.

### TECHNICAL FIELD

The subject matter of the invention is generally related to stress monitoring.

### BACKGROUND

According to the American Institute of Stress, 120,000 people die yearly due to work-related stress. Additionally, healthcare costs resulting from work-related stress total an average of $190 billion annually.

While there are many ways to deal with stress, such as relaxation exercises and calming stimuli, these interventions are not helpful if a person cannot identify that they are stressed.

Unfortunately, measuring stress directly is difficult without cumbersome sensors or invasive methods usually seen in a clinical setting. Stress levels can be assumed from simple measurements such as heart rate, blood pressure, and body temperature, but not all persons' stress responses are alike. Comparing individual biometrics to all other people may result in accurate results.

### SUMMARY

In one embodiment, a method for recommending activities to decrease stress is provided. The method may include monitoring biometric data regarding a user, the biometric data may be captured by a wearable sensor worn by the user; identifying a stress response level based on a comparison of the monitored biometric data to data from a cohort having one or more similarities to the user; generating an activity recommendation based on a speed, degree, and/or duration of one or more changes to the stress response level; and sending a notification to a user device of the user regarding the identified stress response level in real-time, wherein the notification may include the activity recommendation. The method may further include predicting a future stress event based on one or more known stressors and historical data. The method may further include providing a report to the user device, the report regarding one or more of stress response levels, stress performance over time, and/or an effectiveness of the activity recommendation at changing the stress response level.

### BRIEF DESCRIPTION OF THE DRAWINGS

Having thus described the subject matter of the invention in general terms, reference will now be made to the accompanying drawings, which are not necessarily drawn to scale, and wherein:

FIG. 1 illustrates an example of a system for biometric data-based activity recommendation, according to an embodiment of the invention.

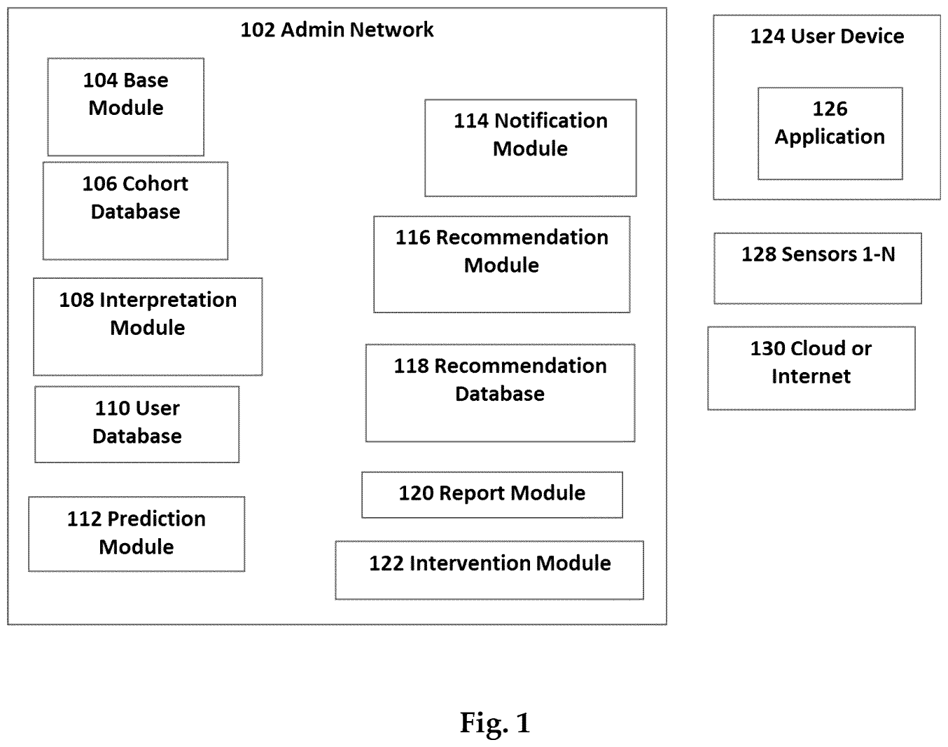

FIG. 2 illustrates an example of a base module, according to an embodiment of the invention.

FIG. 3 illustrates an example of an interpretation module, according to an embodiment of the invention.

FIG. 4 illustrates an example of a user database according to an embodiment of the invention.

FIG. 5 illustrates an example of a prediction module, according to an embodiment of the invention.

FIG. 6 illustrates an example of a notification module, according to an embodiment of the invention.

FIG. 7 illustrates an example of a recommendation module, according to an embodiment of the invention.

FIG. 8 illustrates an example of a recommendation database, according to an embodiment of the invention.

FIG. 9 illustrates an example of a report module, according to an embodiment of the invention.

FIG. 10 illustrates an example of a intervention module, according to an embodiment of the invention.

### DETAILED DESCRIPTION

Embodiments of the subject matter of the invention will be described more fully hereinafter with reference to the accompanying drawings in which like numerals represent like elements throughout the several figures and in which example embodiments are shown. However, the claims' embodiments may be embodied in many different forms and should not be construed as limited to the embodiments set forth herein. The examples set forth herein are non-limiting examples and are merely examples among other possible examples.

The subject matter of the invention may include a method for identifying and quantifying how an individual's stress changes include three elements, the speed, degree, and duration of stress increase (STRESS RESPONSE) caused by STRESSORS (people, places, times, and activities) and STRESS EVENTS (combinations of people, places, and activities), as well as how their stress response changes when they encounter the same stressor or stress event in the future (their STRESS PERFORMANCE) by measuring biometrics that correlate with stress (BIOMETRIC BUNDLE) using non-invasive digital devices with elements that sense all changes but also note when the biometrics change to preset TRIGGER LEVELS (e.g. heart rate +25% above normal) and then automatically record the environment-external factors at that time such as a sound recording, a photograph, and use these to remind the individual later about the stressors involved in those PEAK STRESS MOMENTS, using algorithms, AI and/or ML to classify individuals into different TYPES or cohorts based on these factors, and administering a test that is repeated over time using simple fast questions about their STRESS or CALM IQ—knowledge about their personal stressors, events, biometric bundle, and time.

This is a system for biometric data-based activity recommendation. This system may include an admin network 102, which may be a computer or network of computers. The admin network 102 may include modules and databases useful to the system's functioning. The system may further may include a base module 104, which may collect data from one or more sensors 128, allow the user to interact with the system, and call the other modules at the correct time.

The system may further include a cohort database 106, which may include information about a specific subject group related by a factor such as a time, geographic location, or event. A cohort study may collect and record this data about the subject group. Often, these studies may examine the same cohort group over a long period of time. Cohort data is useful for tracking trends or changes among a population subset or cohort group.

The system may further include an interpretation module 108, which may interpret data from sensors 128. Interpretation may refer to the conversion of raw data into data that can be used by the system. For example, heart rate data, EKG data, body temperature, and/or other like data may be converted into activity level data, heart symptoms data, stress level data, and/or the like. The interpretation module 108 may use data in the cohort database 106 to find relationships between raw data and data that cannot be gathered from the sensors, such as the correlation between heart rate and stress level. Interpreted data may be fed into the interpretation module 108 for further interpretation. For example, the raw data may be converted into stress level data, which may then be reinterpreted in the context of other data to determine if a user is stressed due to exercise, social interaction, physical pain, etc. The interpretation module 108 may use the time to relax, duration of relaxed state relaxation index, time to be stressed, duration of a stressed state, and/or the stress index when making interpretations.

The system may further include a user database 110, which may contain raw sensor data, interpreted sensor data, context data, and/or associated timestamps for a user or users. Examples of data may include, but is not limited to, heart rate data, stress level data, geolocation data, and/or activity data.

The system may further include a prediction module 112, which may predict future events the user may find themselves in the future using the information from the user or user device 124, such as a digital calendar. Events may include, but are not limited to, information such as the place of the event, people who will be at the event, activity the user or others may engage in at the event, and/or the time of the event.

The system may further may include a notification module 114, which may notify the user when the user is stressed. The notification may be sent via the user device 124. The notification may request the user to enter the names of the people involved in the stressful event and the reason for the event. This information may then be saved in the user database 110.

The system may further include a recommendation module 116, which may recommend an activity and/or course of action for the user. For example, the recommendation module 116 may recommend that the user meditate or play calming music if the user is stressed or if the prediction module 112 predicts the user will be in a stressful situation. These recommendations may be retrieved from the recommendation database 118.

The system may further include a recommendation database 118, which may contain a list of recommendations and associated conditions. For example, a recommendation may be guided meditation, or breath training, and the associated condition may be long-duration stress, or a recommendation may be audiobook stories that may help with sleeping. The associated condition may be low-quality or not enough sleep.

The system may further include a report module 120, which may provide a detailed report to the user about their condition based on raw and interpreted data. The report may contain a stress/relaxation chart that shows users the amount of time, e.g., number of hours, they were stressed during the given period. A calm chart may show the user the minimum time it took to relax while using the interventions. The intervention that would result in the least duration for relaxation is the most effective intervention method for the user. A calm print that shows the relaxation response time for different interventions varies and is unique to a user. The response to intervention may be evaluated using the time to reach relaxation, the magnitude of relaxation, and/or the duration which the relaxation level lasted. For each event, the best intervention may be identified. Users may also access these interventions anytime manually. Stress and relaxation responses may be recorded to improve recommendation system. A stress chart may show users when, where, and/or with whom the user was most stressed. A stress print that shows the response for different stress inducers may vary and are unique to a user. The responses to stress may be evaluated using the time to reach relaxation, the magnitude of relaxation, and/or the duration of the relaxation level. A prediction report showing future stressful events and interventions may work best for the user or similar users. Comparison charts may provide the user with information about their average stress level, calm print, and/or stress print compared to, for example, other professions, different age groups, and/or cohorts.

The system may further include an intervention module 122, which may allow the user to initiate an intervention without a trigger. The intervention module 122 may monitor the stress and calm levels of the user and may relate the user's reaction to the intervention delivered, generate a report to the user, and/or update the system recommendations for the user and cohorts of similar users.

The system may further include a user device 124, a mobile device, and/or a wearable device allowing the user to receive recommendations. The user device may transmit data from the sensors 128 to the admin network 102.

The system may further include an application 126, which may be a software application installed on the user device 124. The application may allow the user of the user device 124 to access the admin network 102. A new user may need to sign up and register with the admin network 102 to access services offered and data from the admin network 102. A user may connect a sensor 128 to the application so that the application can communicate and receive data from the sensor 128. The application 126 may direct the user device to connect to, for example, service professionals, such as, for example, a stress coach and/or the like. The application 126 may trigger an automated wearable response, such as a wearable on your ear that emits a sound or an electric pulse designed to relieve stress. Users may maintain a digital journal which may be provided in the mobile app. Further, the information on stressful events (e.g., time, person, place, activity+average HR, and/or stress level) may be added to the journal automatically according to the chronological order. If the user could not enter the person and/or activity details, the journal may provide daily notifications to encourage the user to complete the event details. Users may earn coins, points, and/or the like, for example, by playing quiz games, breathing games, maintaining a journal, and/or the like. During these interventions, users stress and relaxation levels may be monitored, and data obtained may be used to improve the recommendation system, through for example machine learning. Users may receive coins according to the number of games, quizzes, interventions engaged, frequency, and/or duration. Furthermore, users may receive coins for completing journal entries improving the overall engagement. A leaderboard may allow users to check their improvements compared to other users. A wallet may allow the user to see how many coins the user has collected so far.

The system may further include one or more sensors 128, which may measure biometric data and/or other relevant data to be transmitted to the admin network 102. A sensor 128 may be part of a wearable device such as a smartwatch, smartphone, and/or the like. A sensor 128 may be detached from the user device 124, but may, for example, be linked to the device wirelessly. A sensor 128 may transmit data directly to the admin network 126 via the cloud or internet 130. Examples of sensors 128 that detect biometric data may include, but are not limited to, heart rate monitors, pedometers, blood oxygen sensors, thermometers, humidity sensors, and/or the like.

The system may further include a cloud or internet 130, which may be a wired and/or a wireless communication network. The communication network, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, Bluetooth, BLE, and other communication techniques known in the art. The communication network may allow ubiquitous access to shared pools of configurable system resources and higher-level services that can be rapidly provisioned with minimal management effort, often over the Internet, and relies on sharing of resources to achieve coherence and economics of scale, like a public utility, while third-party clouds enable organizations to focus on their core businesses instead of expending resources on computer infrastructure and maintenance.

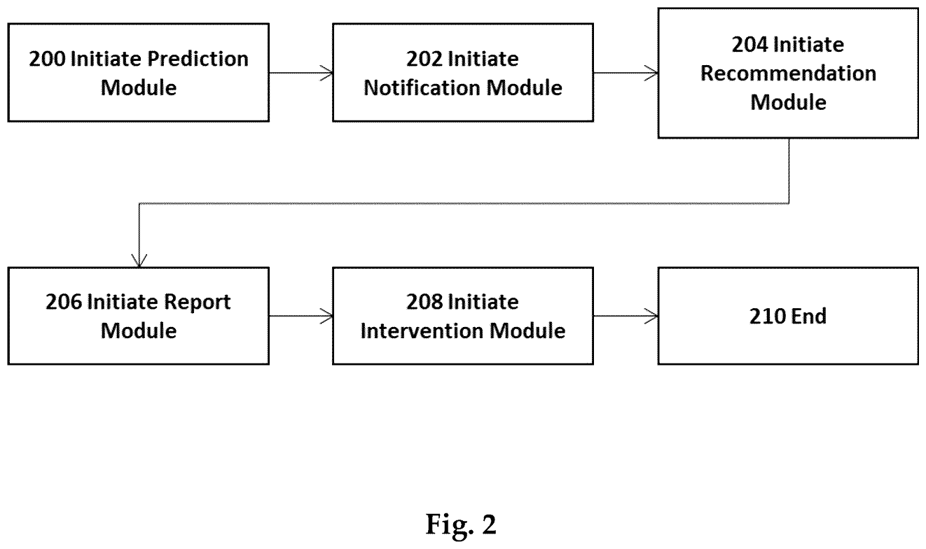

FIG. 2 displays the functioning of the base module 104. The process may begin with the base module 104 initiating the prediction module 112. The prediction module 112 may predict future events, times, interactions and/or activities that may cause the user to feel stressed or some other negative feeling or emotion at step 200. The base module 104 may initiate the notification module 114. The notification module 114 may notify the user when they are stressed and/or when an upcoming event may cause them to be stressed at step 202.

The base module 104 may initiate the recommendation module 116, which may recommend activities and/or techniques to help the user combat stress or other negative emotions and/or feelings at step 204. The base module 104 may initiate the report module 120. The report module 120 may generate a detailed report of the user's status for the user to view at step 206. At any point in the process the user may initiate the intervention module 122 at step 208. The base module 104 may end at step 210.

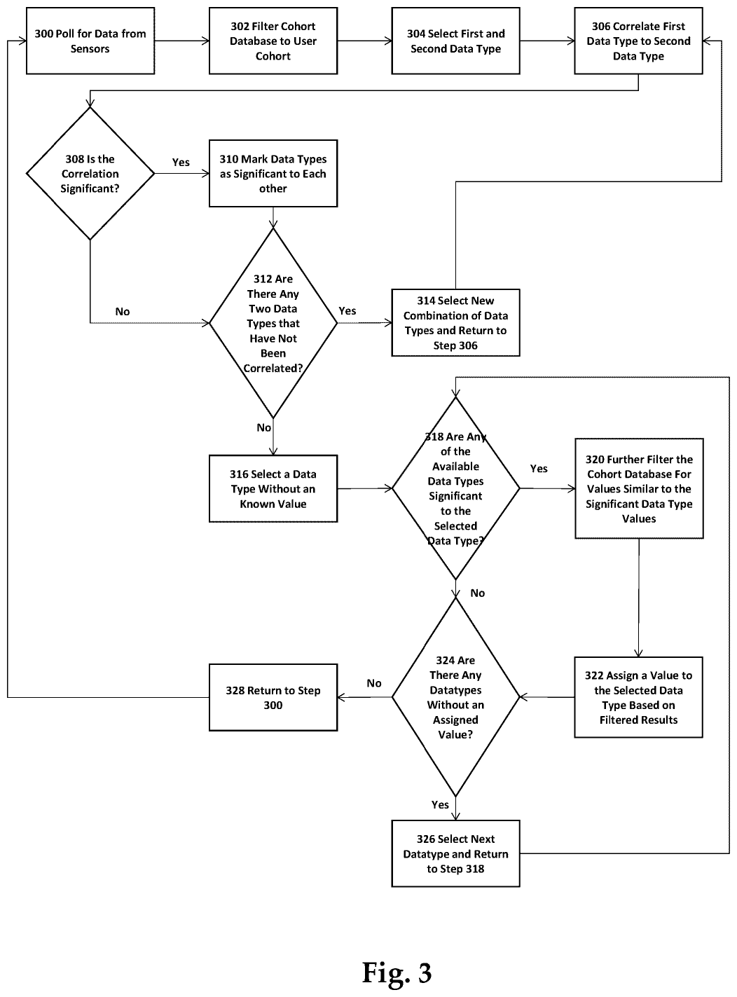

FIG. 3 displays the functioning of the interpretation module 108. The process may begin with the interpretation module 108 polling for data from the sensors 128. Data may be received continuously from the sensors 128. Data may be received from more than one sensor 128 concurrently. The interpretation module 128 may collect data over some time, such as a minute, before moving on to the next step at step 300.

The interpretation module 108 may filter the cohort database 106 to a cohort or cohorts of persons who share similarities with the user. Cohorts may be defined by, but not limited to, professional categories such as teachers, engineers, and/or doctors, age categories such as, but not limited to, 20-24 years, 25-29 years, 30-34 years, etc., sex and/or gender categories, symptom categories, etc. For example, if the user is a male teacher aged 31, then the cohort database may be filtered to only records of persons who are also male teachers and between the ages of 30-34. The cohort may be expanded if such filtering leaves too few or no data records. For example, the previous example cohort may be expanded to include teachers of any sex or gender, teachers older than 34, or males in similar professions such as coaches and tutors. User identifying information may be provided by the user to increase the accuracy of their interpreted data at step 302.

The interpretation module 108 may select a first and second data type from the filtered cohort data. The data type may refer to the measurement being made. Example data types may include, but are not limited to, temperature data, heart rate data, stress level data, symptom data, activity level data, time data, and/or location data at step 304.

The interpretation module 108 may calculate a correlation coefficient for the two data types. The interpretation module 108 may use Pearson correlation or other correlation methods well-known in the art. In an example of highly correlated data, the correlation coefficient for heart rate and stress level may be 0.81. In another example of uncorrelated data, the correlation coefficient for body temp and stress level may be 0.13 at step 306.

The interpretation module 108 may determine if the correlation coefficient is above a predetermined threshold, for example, 0.75, in order to determine if the data is highly correlated and deemed a relevant correlation. The threshold may be static or dynamic and be set by an administrator of the system, a system user, or another module. If the correlation coefficient is less than the threshold, the interpretation module 108 may skip to step 312 at step 308.

If the correlation coefficient is above a predetermined threshold, the interpretation module 108 may mark the two selected data types as significantly correlated to each other at step 310. The interpretation module 108 may determine if there are any combinations of two data types that have not had a correlation coefficient calculated. For example, the interpretation module may have calculated a correlation coefficient between, for example, heart rate and stress level, but not sweat intensity and stress level at step 312.

If any combinations of, for example, two data types have not had a correlation coefficient calculated, the interpretation module 108 may select the next combination of data types and return to step 306 at step 314. If all combinations of data types have had a correlation coefficient calculated, the interpretation module 108 may select a data type without a known value for the current user. For example, stress levels may not be a data type that can likely be recorded by a wearable sensor. Thus, the user's stress level may be unknown unless it has been interpreted from other data. Selection of a data type at this step may deselect any currently selected data at step 316.

The interpretation module 108 may determine if any of the data available is of a data type that is significant to the selected data type. For example, the selected data type is stress level. Stress level may be significantly correlated to heart rate. Data from the sensors 128 may indicate, for example, the user has a heart rate of 62 bpm. Therefore there is available data of a data type that is significant to the selected data type. Available data may include data not directly from the sensors 128, such as medical history. For example, if there is medical data on a user that indicates they have a high baseline heart rate, high blood pressure, type 2 diabetes, and/or any other medical condition that does not vary from day to day, the interpretation module 108 may determine that this data is available. Thresholds can be configured using this data. If there is no data available of a data type that is significant to the selected data type, the interpretation module 108 may skip to step 324 at step 318.

If any of the data available is of a data type that is significant to the selected data type, the interpretation module 108 may further filter the cohort database 106 based on the values of the significant data types. For example, heart rate and body temperature may be significant data types. In an example, the user's heart rate may be 62 bpm, and body temperature may be 96.9ºF. The cohort database 106 would be filtered to only results that match these two values. The match may be an exact match or within a range of values, such as 60-65 bpm at step 320.

The interpretation module 108 may assign a value to the selected data type based on the filtered results. For example, the cohort database may have been filtered down to 100 results. Of those results, the average stress level may be 13 on the perceived stress scale, which goes, for example, from 0 to 40. This average may be assigned as the user's stress level value. The assigned value may be the mean, median, mode, and/or some other statistical value representing the combined values of the filtered results at step 322.

The interpretation module 108 may determine if there are any data types without an assigned value. Some data types may never receive an assigned value due to insufficient data from the sensors 128 or in the cohort database 106 and may be skipped once they have been already selected at step 324. If there are data types without an assigned value, the interpretation module 108 may select the next data type and return to step 318 at step 326. if there are no more data types without an assigned value, the interpretation module 108 may return to step 300 at step 328.

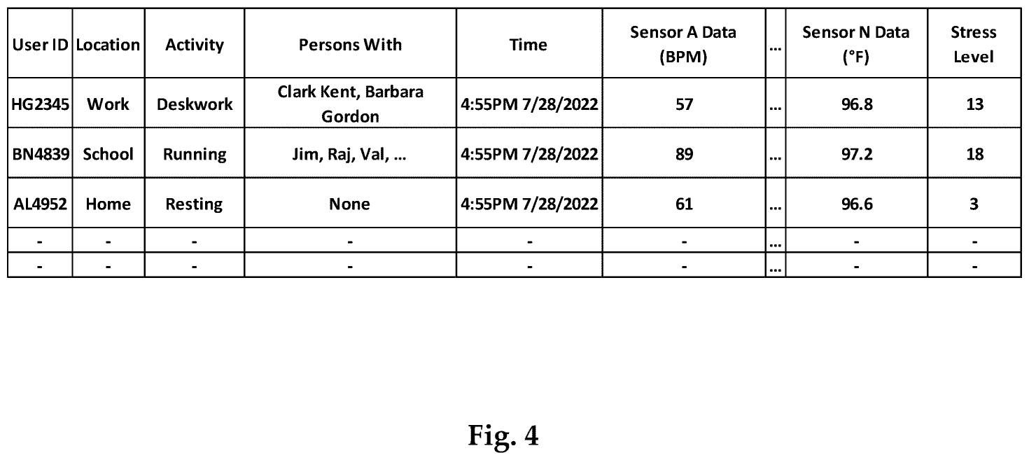

FIG. 4 displays an example of user database 110. The user database 110 may contain raw sensor data, interpreted sensor data, context data, associated timestamps, and/or other like data for a user or users. Examples of data may include, but limited to, heart rate data, stress level data, geolocation data, activity data, and/or other like data. Data in the user database 110 may be user-defined. For example, a user may indicate through the application 126 that a certain location is a home, work, or school. A user may define which persons they are with directly, or these names and identities may be taken from the user's contacts.

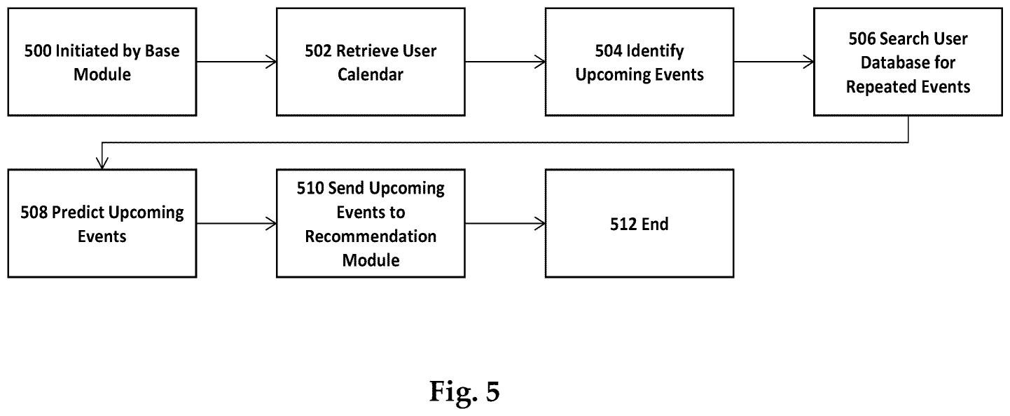

FIG. 5 displays the functioning of the prediction module 112. The process may begin with the prediction module 112 being initiated by the base module 104 at step 500. The prediction module 112 may retrieve the user's calendar information if available. The user device 124 may be a smartphone with a calendar application or other suitable device. The user may have an online calendar from which data may be retrieved via the cloud or internet 130. There may be user calendar information in the user database 110 or another database on the admin network 102. If no user calendar data is available, the prediction module may skip to step 506 at step 502.

The prediction module 112 may identify upcoming events. Which calendar data is an upcoming event may, for example, be identified by the functionality of the calendar. For example, the calendar may indicate which calendar entries are events and which are other entries such as personal reminders. The prediction module 112 may use natural language processing to identify events. For example, a calendar entry for “John's Birthday Party” may be recognized as an event from the title, whereas “replace water filter” may not be identified as an event. This identification method may be tailored to each user and improve over time with feedback and/or machine learning. The location, persons present, activity, and/or other information associated with the event may be identified if that data is included in the calendar or can be identified using natural language processing at step 504.

The prediction module 112 may search the user database for repeated events for the same user. An example of a repeated event may be a Tuesday morning meeting that occurs regularly at step 506. The prediction module 112 may predict upcoming events by assuming that a repeated event may continue to follow the same pattern. For example, a meeting that occurred almost every Tuesday morning would be predicted to occur next Tuesday. The prediction module 112 may predict, for example, the same location, persons present, and activity as previous events at step 508. The prediction module 112 may send the identified and predicted upcoming events to the recommendation module 116 at step 510. The prediction module 112 may end at step 512.

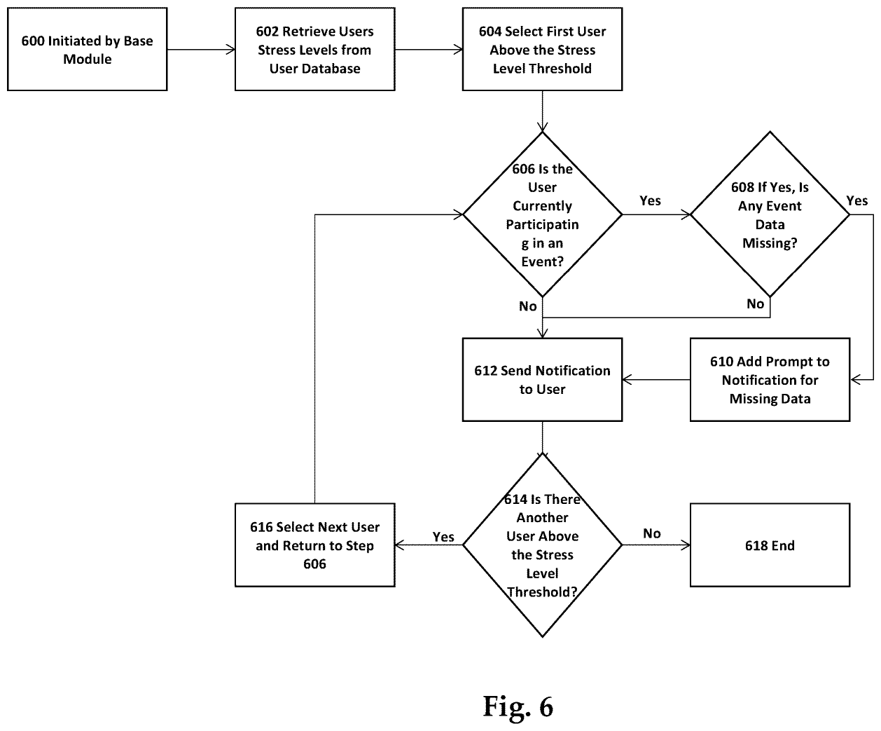

FIG. 6 displays the functioning of the notification module 114. The process may begin with the notification module 114 being initiated by the base module 104 at step 600. The notification module 114 may retrieve user stress levels from the user database 110 at step 602. The notification module 114 may select the first user above a stress level threshold, such as, for example, a stress level of 15, which may be a moderate level of stress. The stress level threshold may be static or dynamic and may be set by an administrator of the system, a system user, or another module at step 604.

The notification module 114 may determine if the user is currently participating in an event. The notification module 114 may retrieve this information from the user database 110, the user device, a user's calendar data, etc. If there is no current event, the notification module 114 may skip to step 612 at step 606. If the user participates in an event, the notification module 114 may determine if any event data is missing, such as the location, persons present, and/or activity the user is participating in. For example, the event may be a work meeting, but the system may not yet have data on where the work meeting is happening and who else is in attendance. If no event data is missing, the notification module 114 may skip to step 612 at step 608. If there is event data missing, the notification module 114 may add a prompt to the notification for the missing information. For example, the notification may include “Who are you with?” or “What are you doing right now?” with a space to respond at step 610.

The notification module 114 may send a notification to the user. The notification may include that the user is stressed. For example, “Your stress level is moderate.” The notification may include recommendations from the recommendation module 116 if available. The notification may include recommended interventions, or options to auto-activate pre-selected interventions. The notification may be sent to the user device 124 via SMS or email or directly to the application 126, which may display the notification or otherwise notify the user, such as with sound, haptic feedback, or other notification type at step 612. The notification module 114 may determine if another user is above the stress level threshold at step 614. If another user is above the stress level threshold, the notification module 114 may select the next user and return to step 606 at step 616. The notification module 114 may end at step 618.

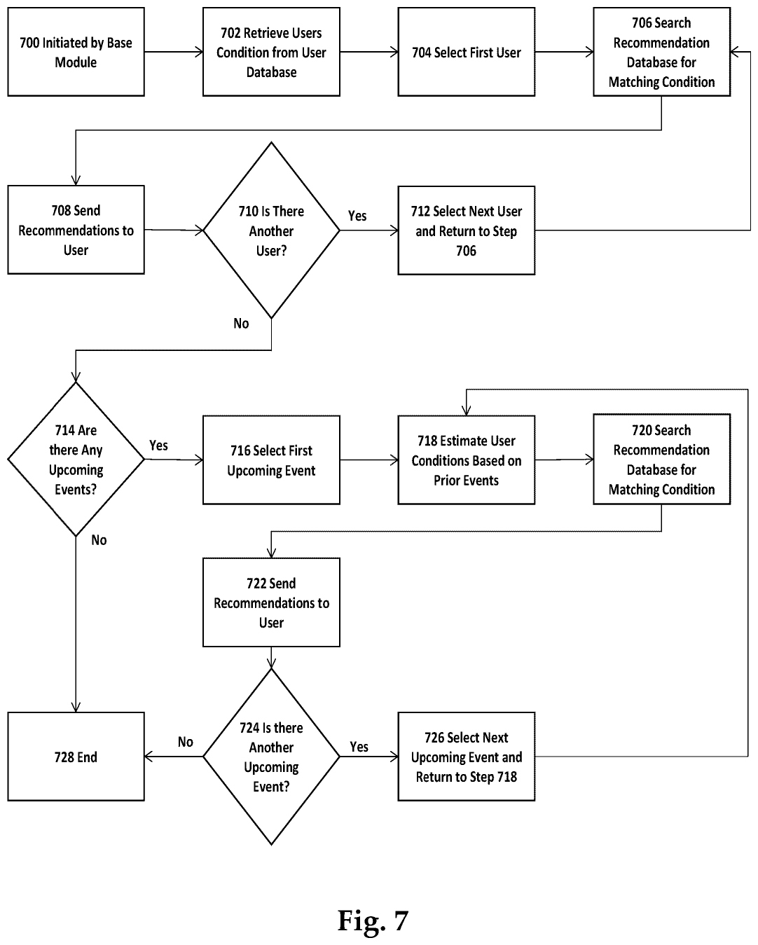

FIG. 7 displays the functioning of the recommendation module 116. The process may begin with the recommendation module 116 being initiated by the base module 104 at step 700. The recommendation module 116 may retrieve user's or users' conditions from the user database 110. User conditions may include any biometric data from the sensors 128 or the interpretation module 108, such as stress level, activity level, heart rate, etc. The condition may also refer to derivatives of these biometrics. For example, speed of change in stress level, duration of elevated or depressed stress level, and/or the degree of the change in the stress level at step 702.

The recommendation module 116 may select a first user at step 704. The recommendation module 116 may search the recommendation database 118 for conditions that match the conditions of the selected user. Matching may refer to a range of values. For example, if the condition in the recommendation database 118 is a stress level above, for example 10, then a stress level of 12 would match because it is greater than 10. Conditions may include more than one biometric, such as for example stress level and heart rate, which may be required to match or alternative ways to match. Conditions may also refer to derivatives of these biometrics. For example, speed of change in stress level, duration of elevated or depressed stress level, and/or the degree of the change in the stress level at step 706.

The recommendation module 116 may send the matching recommendations to the user. The recommendation module 116 may send a subset of these recommendations, such as, but not limited to, the top three (3) closest matches. The recommendation module 116 may not recommend activities previously shown to be ineffective for the user or that the user has rejected in the past at step 708. The recommendation module 116 may determine if there is another user at step 710. If there is another user, the recommendation module 116 may select the next user and return to step 706 at step 712. If there is not another user, the recommendation module 116 may determine if there are any upcoming events from the prediction module 112. If there are no upcoming events, the recommendation module 116 may skip to step 728 at step 714.

The recommendation module 116 may select the first upcoming event from the prediction module 112 at step 716. The recommendation module 116 may estimate the condition of the user at the predicted event based on previous events in the user database 110. For example, if the user is going to a regular work meeting, it may be estimated that the user's condition may be the same as in previous work meetings. The estimated condition may be adjusted based on changes to the location, persons present, event activity, and/or other information at step 718.

The recommendation module 116 may search the recommendation database 118 for conditions that match the estimated conditions of the user for the selected upcoming event. Matching may refer to a range of values. For example, if the condition in the recommendation database 118 is a stress level above 10, then a stress level of 12 would match because it is greater than 10. Conditions may include more than one biometric, such as, but not limited to, stress level and heart rate, which may be required to match or alternative ways to match. Conditions may also refer to derivatives of these biometrics. For example, speed of change in stress level, duration of elevated or depressed stress level, and/or the degree of the change in the stress level at step 720.

The recommendation module 116 may send the matching recommendations to the user. The recommendation module 116 may send a subset of these recommendations, such as, but not limited to, the top three (3) closest matches. The recommendation module 116 may not recommend activities previously shown to be ineffective for the user or that the user has rejected in the past. The recommendation module 116 may also account for the expected future time of the event by recommending activities that may take longer and deal with future conditions instead of short activities that deal with immediate conditions at step 722. The recommendation module 116 may determine if there is another upcoming event at step 724. If there is another upcoming event, the recommendation module 116 may select the next upcoming event and return to step 718 at step 726. The recommendation module 116 may end at step 728.

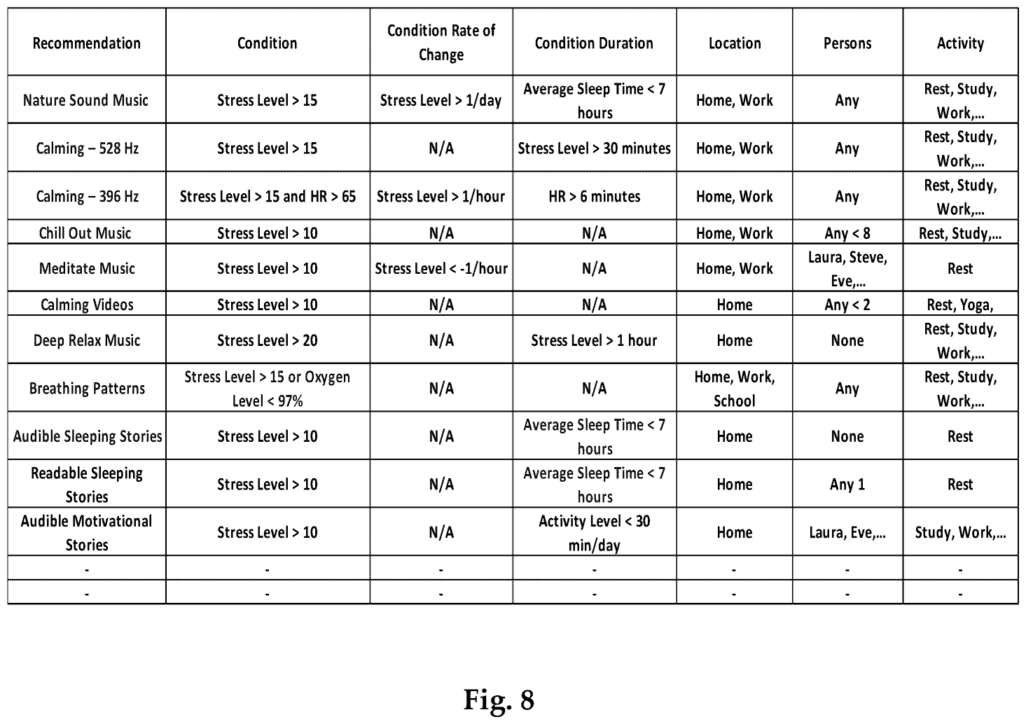

FIG. 8 displays the recommendation database 118. The recommendation database 118 may contain a list of recommendations and associated conditions. In one non-limiting example, a recommendation may be guided meditation, and the associated condition may be long duration stress, or a recommendation may be audiobook stories that may help with sleeping. The associated condition may be low-quality or not enough sleep. The associated condition may include one or more data types in one or more combinations. The associated conditions may be uniquely tailored to a user. A user may be able to reject recommendations, and a record of that rejection may be kept in the recommendation database 118.

The data in the recommendation database 118 may be adjusted by a machine learning algorithm based on the effectiveness of a recommendation, and/or whether a user rejected the recommendation. For example, the recommendation “meditate music” may be effective at low stress levels, but ineffective or even counterproductive at high stress levels. The conditions associated with “meditate music” may be altered to reflect this effective range, such as “20>stress level>10” or “stress level>10 and stress level<20”. For another example, the recommendation “nature sound music” may be ineffective or consistently rejected while the user is at work. This may be due to the user's work not allowing music or being too loud for music to be effective. The locations associated with “nature sound music” may be altered to no longer include the user's workplace.

The effectiveness of a recommendation on stress levels may take into account the magnitude of the reduction in stress level, the duration of the reduction in stress level, the speed of the reduction in stress level, etc. Adjustments may be made based on an individual user, a set of similar users, all users, or any combination of these options. Some recommendations may not require user input and may begin automatically. For example, “Calming—528 Hz” may refer to tones which may be played from the speakers of the user device 124. These tones may be played automatically whenever the user matches one or more of the associated parameters in the recommendation database 118. The type, duration, and intensity of tones played may be adjusted by the machine learning algorithm to maximize reduction in stress level. For example, once the user has dropped to a stress level of 8 the tones are no longer effective and may be shut off. Further, the machine learning algorithm may learn which recommendations may be started automatically and which may be first accepted by the user.

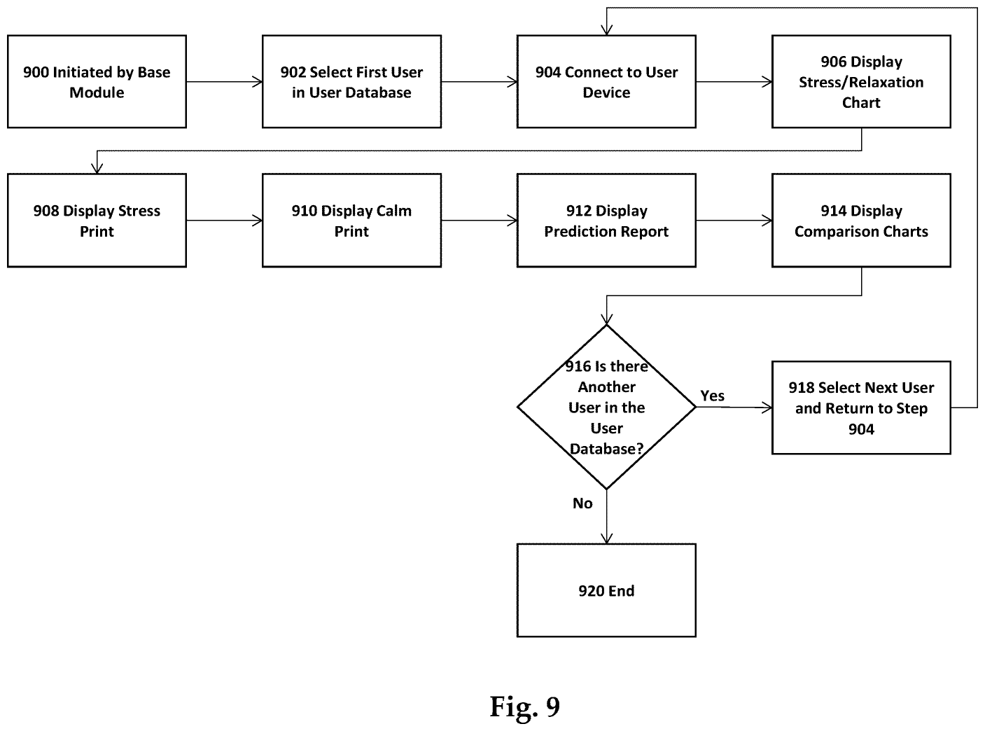

FIG. 9 displays the functioning of the report module 120. The process may begin with the report module 120 being initiated by the base module 104. The base module 104 may also be initiated by the user via the user device 124 or application 126 at step 900. The report module 120 may select the first user in the user database 110. If the report module was initiated by a user, then only that user may be selected at step 902. The report module 120 may connect to the user device 124 of the selected user at step 904.

The report module 120 may display a stress/relaxation chart showing users the amount of hours they were stressed during the given time. The chart may be separated into a relaxation chart which may show the user the minimum time it took them to relax while using the interventions, and a stress chart which shows users when, where, and with whom the user was most stressed. The chart or charts may display data from the user database 110. The chart or charts may not be displayed until the user opens the application 126 or navigates to the page within the application 126, where reports would be displayed at step 906.

The report module 120 may display a stress print showing that the response for different stress inducers varies and is unique to a user. The responses to stress may be evaluated using the time to reach relaxation, the magnitude of relaxation, and/or the duration of the relaxation level. The stress print may display data from the user database 110. In one example, the stress print may not be displayed until the user opens the application 126 or navigates to the page within the application 126, where reports would be displayed at step 908.

The report module 120 may display a calm print which may show the relaxation response time for different interventions varies and is unique to a user. The response to intervention may be evaluated using the time to reach relaxation, the magnitude of relaxation, and/or the duration of the relaxation level. For each event, the best intervention may be identified. The calm print may display data from the user database 110. In one example, the calm print may not be displayed until the user opens the application 126 or navigates to the page within the application 126, where reports would be displayed at step 910.

The report module 120 may display a prediction report showing future stressful events and interventions that seem to work best for the user or similar users. The prediction report may display data from the prediction module 112. In one example, the prediction report may not be displayed until the user opens the application 126 or navigates to the page within the application 126, where reports would be displayed at step 912.

The report module 120 may display comparison charts in which the user may be provided with information about his average stress level, calm print, and/or stress print compared to, for example, other professions, different age groups, and/or cohorts. The charts may display data from the user database 110. In one example, the charts may not be displayed until the user opens the application 126 or navigates to the page within the application 126, where reports would be displayed at step 914. The report module 120 may determine if there is another user in the user database 110 for which reports have not been generated at step 916. If there is another user, the report module 120 may select the next user and return to step 904 at step 918. If there is no other user, the report module 120 may end at step 920.

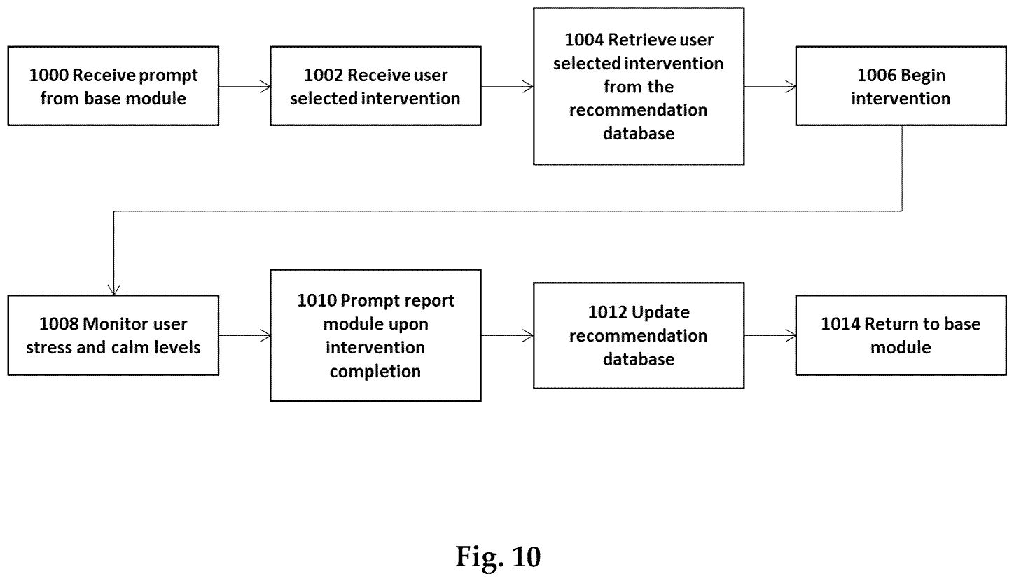

FIG. 10 illustrates the intervention module 122. The process may begin with the intervention module 122 being initiated by a prompt from the base module 104 at step 1000. The prompt from the base module 104 may be triggered by a user selection of an intervention from the user device 124. The user selected intervention may be received at step 1002.

The user selected intervention may then be retrieved from the recommendation database 118. The retrieved intervention may be initiated at step 1006. The user's response, such as their stress or calm level, to the intervention may be monitored at step 1008 through the user device 124 and/or the sensors 128. Upon completion of the intervention the report module 120 may be prompted at step 1010. The user's response may then be used to update the recommendations database 118 at step 1012. The process may then return to the base module 104 at step 1014.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the subject matter of the present invention. For example, the term “about,” when referring to a value can be meant to encompass variations of, in some embodiments ±100%, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for recommending activities to decrease stress, the method comprising:
a. monitoring biometric data regarding a user, the biometric data captured by a wearable sensor worn by the user;
b. identifying a stress response level based on a comparison of the monitored biometric data to data from a cohort having one or more similarities to the user;
c. generating an activity recommendation based on one or more of a speed, degree, and/or duration of one or more changes to the stress response level; and
d. sending a notification to a user device of the user regarding the identified stress response level in real-time, wherein the notification includes the activity recommendation.

2. The method of claim 1, further comprising predicting a future stress event based on one or more known stressors and historical data.

3. The method of claim 1, further comprising providing a report to the user device, the report regarding one or more of stress response levels, stress performance over time, and an effectiveness of the activity recommendation at changing the stress response level.
